(19) United States
(12) Patent Application Publication
(10) Pub. No.: US 2008/0194544 A1
(43) Pub. Date: Aug. 14, 2008

(54) AQUEOUS FORMULATIONS OF EPINASTINE FOR TREATING ALLERGIC RHINITIS

(76) Inventor: RAMESH KRISHNAMOORTHY, Cary, NC (US)

Correspondence Address: HOWERY LLP, C/O IP DOCKETING DEPARTMENT, 2941 FAIRVIEW PARK DRIVE SUITE 200, FALLS CHURCH, VA, 22042, US

(21) Appl. No.: 11/770,383

(22) Filed: Jun. 28, 2007

Related U.S. Application Data
(60) Provisional application No. 60/888,926, filed on Feb. 8, 2007.

(51) Int. Cl. A61K 31/55; A61P 37/00

(52) U.S. Cl. 514/214.02

(57) ABSTRACT

There is provided homogeneous pharmaceutical compositions for the treatment of, for example, rhinitis, asthma and/or chronic obstructive pulmonary disease comprising a corticosteroid and an antihistamine, a polar lipid liposome and a pharmaceutical-acceptable aqueous carrier.

This application claims the benefit of U.S. Provisional Application No. 60/888,926, filed Feb. 8, 2007; which is incorporated herein by reference in its entirety.

### TECHNICAL FIELD

This invention relates to pharmaceutical formulations, particularly aqueous nasal preparations, of an antihistamine compound, such as epinastine hydrochloride and related compounds. This invention also relates to methods of treating allergic rhinitis by intranasal delivery of a small volume of a viscous epinastine formulation, whereby the patient perceives a minimal or no bitter taste of epinastine.

### BACKGROUND OF THE INVENTION

Epinastine, chemically known as 3-amino-9,13b-dihydro-1H-dibenz-[c,-f]imidazol[1,5-a]azepine, and its acid addition salts are disclosed in German Patent application P 30 08 944.2 which forms the basis for EP 0035749. Methods for the preparation of epinastine are described in EP 0496306 or WO 01/40229. Epinastine is most often used for its antihistaminic effects.

Epinastine hydrochloride (ELASTAT®) has been approved as an eye drop in U.S. for treating allergic conjunctivitis. Epinastine hydrochloride has been approved as an oral tablet in Japan and some South American countries for treating allergic rhinitis. However, especially for children and elderly people, tablets are not always easy to take. It is found that aqueous formulations of epinastine-hydrochloride result in bad taste, reported as bitterness or bitter aftertaste. The bitter taste of epinastine is strong and could not be masked by the use of a single conventional taste-masking agent such as sucrose.

ASTELIN® (0.1% azelastine hydrochloride) nasal spray was approved for treating allergic rhinitis in the United States. When ASTELIN® was administered to subjects at 137 μL per spray, two sprays per nostril, twice daily, 19.7% of subjects reported adverse effects of bitter taste (see product package insert of ASTELIN® nasal spray).

US2003/0104017 discloses a pharmaceutical formulation comprising epinastine or an acid addition salt thereof, and at least two kinds of sweeteners or flavoring agents, wherein one of the at least two kinds of sweeteners or flavoring agents masks the quick-acting bitterness of epinastine or its salt and the other one masks the long-acting bitterness of epinastine or its salt.

WO2004/075900 discloses pharmaceutical powder formulation comprising epinastine, sodium lauryl sulfate, sweetening agents and/or flavoring agents.

US2003/0050303 discloses a method for inhibiting the influx of neutrophils and eosinophils into the tissue of the ocular conjunctiva or the nasal mucous membrane in a host, the method comprising topically administering to a host in need of such treatment an aqueous solution comprising epinastine, optionally in the form of its racemate, its enantiomers, or its pharmacologically acceptable acid addition salts thereof, in a concentration of 0.005 to 0.5 mg/ml of solution.

US2002/037297 discloses a process for the topical treatment of or prophylaxis against allergic rhinitis, vasomotoric rhinitis, conjunctivitis, cold, cold-like and/or flu symptoms. The method comprises topically administering to mucous tissues of a patient in need therefor a non-sedating antihistamine and an α-adrenergic agonist.

US2006/0110331 discloses a composition comprising azelastine, hypromellose as a viscosity modifier and sucralose as a taste-masking agent.

Currently, there is not an effective method for treating allergic rhinitis with an aqueous intranasal spray formulation without causing strong and unpleasant bitter taste associated with post-nasal drip. There is a need for an improved aqueous nasal spray formulation for treating allergic rhinitis; such aqueous nasal spray formulation is not only effective to treat allergic rhinitis but also has an acceptable taste profile following repeated dosing.

### SUMMARY OF THE INVENTION

The present invention is directed to an aqueous pharmaceutical formulation comprising epinastine or an acid addition salt thereof, and a viscosity-enhancing agent. The viscosity-enhancing agent provides an enhanced viscosity of the formulation without causing precipitation of the active ingredient epinastine or other ingredients. The increased viscosity of the formulation minimizes post-nasal drip and reduces the possibility of the formulation to ‘drip back’ from the nasal cavity to the back of the throat. By minimizing the postnasal drip, the bitter taste of epinastine is less perceived by the subject. The viscosity-enhancing agent is a polymer selected from the group consisting of hydroxypropylmethylcellulose, methylcellulose, ethylcellulose, hydroxyethylcellulose, hydroxypropylcellulose, polyvinyl alcohol, polyvivnyl pyrrolidone, and the combination thereof.

The aqueous pharmaceutical formulation of the present invention typically comprises 0.05-0.2% (w/v) epinastine or an acid addition salt thereof, 0.05-0.5% (w/v) viscosity-enhancing agent, a buffer to maintain a pH between 5-8, and a tonicity agent to maintain a tonicity between 200-400 mOsm/kG.

The present invention also provides a method for treating allergic rhinitis in a subject. The method comprises the steps of: identifying a subject suffering from allergic rhinitis; administering to the nose of the subject one to two sprays at ≦115 μL per nostril per spray once or twice daily of an aqueous epinastine formulation; wherein said aqueous epinastine formulation comprises (a) 0.05-0.2% (w/v) of epinastine or an acid addition salt thereof, (b) 0.05-0.5% (w/v) of a viscosity-enhancing agent selected from the group consisting of hydroxypropylmethylcellulose, methylcellulose, ethylcellulose, hydroxyethylcellulose, hydroxypropylcellulose, polyvinyl alcohol, polyvinyl pyrrolidone, and the combination thereof, to maintain the viscosity between 1.5-10 centipoise, and (c) a tonicity agent to maintain a tonicity between 200-400 mOsm/kG; whereby the subject perceives a minimal or no bitter taste of said epinastine and the symptoms of allergic rhinitis are reduced. A preferred viscosity-enhancing agent is hydroxypropylmethylcellulose. A preferred tonicy agent is propylene glycol.

### DETAILED DESCRIPTION OF THE INVENTION

The inventor has unexpectedly discovered an aqueous epinastine formulation that can be delivered topically for use in the nose or in the eye and has a minimal or no bitter taste. The aqueous epinastine formulation is viscous and has a viscosity of 1.5-10 centipoises. When intranasally delievered in a small volume of ≦120 μL, preferably ≦115 μL, preferably ≦100 μL, preferably ≦90 μL, more preferably ≦85 μL per nostril per spray, the epinastine formulation of the present invention is effective in treating allergic rhinitis, and does not cause a strong bitter aftertaste, even without including taste-masking agents such as sweeteners or flavoring agents in the formulation. Although the nose has a large space relative to the spray volume emitted by a small volume metered-dose nasal spray pump, the inventor has unexpectedly discovered that reducing the nasal spray volume from 137 μL (ASTELIN® nasal spray volume) to ≦115 μL, preferably to <85 μL, plays an important role in minimizing the bitter aftertaste of epinastine. The inventor has discovered that delivering a small volume of the viscous epinastine formulation of the present invention reduces the bitter aftertaste significantly without compromising the therapeutic efficacy of epinastine. The viscous epinastine formulation is delivered to the nose of a patient using a metered-dose nasal spray pump. The viscous epinastine formulation has minimal post-nasal drip and does not cause an unacceptable quick-acting bitterness or a long-acting bitterness after dosing.

This invention is directed to an aqueous pharmaceutical formulation comprising an antihistamine chemical compound such as epinastine and salts thereof. This invention provides a formulation containing one or more viscosity-enhancing agents that increase the viscosity of the formulation and minimize the bitter taste of epinastine. The formulation does not contain a substantial amount of unacceptable agents for pharmaceutical, particularly, ophthalmic and nasal use. The invention provides a stable aqueous formulation of epinastine; the formulation is suitable for therapeutic uses and remains stable under normal use storage conditions for an extended period of time.

The aqueous pharmaceutical formulations of the present invention exclude the use of inappropriate adjuvants that can cause toxicological outcomes and tissue damage when used in humans or mammals for a long term. The aqueous pharmaceutical formulations of the present invention contain epinastine in solution at sufficient concentrations, and provide an anti-allergic response in mammals. The aqueous pharmaceutical formulations are non-irritating and tolerable to human epithelial cells, and are suitable for multiple instillations.

The present invention is directed to an aqueous pharmaceutical formulation comprising epinastine or a salt thereof, and a viscosity-enhancing agent. Epinastine can be used either as free base or as a pharmaceutically acceptable salt thereof. Preferably, epinastine is used in the form of its acid addition salts such as hydrochloride salt.

“A viscosity-enhancing agent,” as used herein, refers to a compound when added to a solution increases the viscosity of the solution. A viscosity-enhancing agent at a suitable concentration can modify or alter the flow properties of the system from a Newtonian fashion (e.g. water) to a pseudo plastic or plastic flow. An increased viscosity alters the residence time, drainage characteristics, and/or bioavailability of the pharmaceutical formulation. A viscosity-enhancing agent typically increases the viscosity of the pharmaceutical formulation 1.5-10 fold (e.g. 1.5-10 centipoises) with respect to water (approximately 1 centipoise).

The viscosity-enhancing agent used in the formulation provides an enhanced viscosity of the formulation without causing precipitation of the active ingredient epinastine or other ingredients. Furthermore, the viscosity-enhancing agent is compatible with other agents in the formulation. The increased viscosity of the formulation provides a sustained action, minimizes post-nasal drip, and reduces the possibility of the formulation to ‘drip back’ from the nasal cavity to the back of the throat. By minimizing the postnasal drip, the bitter taste of epinastine is less perceived by the subject.

A viscosity-enhancing agent useful for the present invention is often a polymer such as hydroxypropylmethylcellulose, methylcellulose, ethylcellulose, hydroxyethylcellulose, hydroxypropylcellulose, polyvinyl alcohol, and polyvivnyl pyrrolidone. A preferred viscosity-enhancing agent is hydroxypropylmethylcellulose. The viscosity-enhancing agent often improves the physical stability of the formulation. The inventor has discovered that not all typical pharmaceutically acceptable viscosity modifiers are suitable for use in this invention because they are physically incompatible with epinastine in solution and result in flocculation and phase separation, and/or they do not function well at pH 4-8. For example, polyvinyl acrylic acid, polystyrene sulfonic acid, sodium carboxymethylcellulose, xanthan gum, microcrystalline cellulose and sodium carboxymethylcellulose are not suitable for this invention.

Typically, the aqueous pharmaceutical formulation of the present invention comprises 0.001-3% (w/v) epinastine or an acid addition salt thereof, 0.001-0.5% (w/v) viscosity-enhancing agent, a buffer to maintain a pH between 4-8, and a tonicity agent to maintain a tonicity between 200-400 mOsm/kG. The viscosity of the formulation is about 1.5-10 centipoises (cps), preferably 1.5-10 cps, more preferably 2-9 cps, and more preferably 2-6 cps.

The concentration of epinastine in the aqueous formulation is in general 0.001-3%, preferably 0.005-1% or 0.005-0.6%, and more preferably 0.05-0.2% or 0.1-0.15% (w/v). For example, a preferred concentration of epinastine is about 0.1% or about 0.15%.

As used in this application, “about” refers to ±15% of the value recited.

The concentration of a viscosity-enhancing agent in the aqueous formulation is in general 0.001-5%, preferably 0.05-0.5%, and more preferably 0.1-0.3% (w/v).

The pH of the present formulation is 4-8, preferably 5-8, more preferably 6.0-7.5. Buffers suitable to maintain the pH between 4-8 include phosphate, citrate buffer, acetate buffer, maleate buffer, tartarate buffer, or combination thereof. Phosphate buffer or citrate buffer is preferred. For long-term stability, the formulation is preferred to have a pH of 5-8. Buffers suitable to maintain the pH of 5-8 include citrate buffer, phosphate buffer, citrate/phosphate buffer, maleate buffer, tartarate buffer, or combination thereof. A suitable concentration of the buffer is 1-100 mM, preferably 5-50 mM, more preferably 5-25 mM, and most preferably 10-20 mM.

The tonicity agent is present in an amount to achieve a tonicity between 200-400, preferably 220-380, and more preferably 250-340 mOsm/kG. The tonicity agent can be non-ionic or ionic. A non-ionic tonicity agent is preferred because its compatibility with polymeric adjuvant that functions as a viscosity-enhancing agent. Non-ionic tonicity agents include diols, such as glycerol, mannitol, erythritol; and sugars such as sucrose and dextrose. Other non-ionic tonicity agents such as glycerol, polyethylene glycol, propylene glycol, which also function as cosolvents and taste-masking agent, can also be used. The non-ionic tonicity agent is in general in an amount of 1-20%, preferably 1-10%, more preferably 1-5%. Preferred non-ionic agents are mannitol, sucrose, dextrose, propylene glycol, in an amount of 1-5%. For Example, propylene glycol at 1-2% or 1-1.8% (w/v) is a preferred non-ionic tonicity agent for the present invention.

The tonicity agent can also be ionic agents such as sodium chloride, potassium chloride, or a balanced salt solution. The ionic tonicity agents are typically present in an amount of 0.5-0.9%, preferably 0.6-0.9%.

The pharmaceutical formulation of the present invention optionally comprises a chelating agent. A chelating agent is a substance which can form several coordinate bonds to a metal ion. Chelating agents offers a wide range of sequestrants to control metal ions in aqueous systems. By forming stable water-soluble complexes with multivalent metal ions, chelating agents prevent undesired interaction by blocking normal reactivity of metal ions. Ethylenedinitrilotetraacetic acid (EDTA), diethylenetriaminepentaacetic acid (DTPA), and N,N-bis(carboxymethyl)glycine (NTA) are examples of chelating agents for the present inventions. EDTA (ethylenediamine tetraacetate) is a preferred chelating agent. The chelating agents are typically present in an amount of 0.01-1%, and preferably 0.02-0.5% w/v.

Health regulations in various countries require that multi-dose ophthalmic and nasal preparations include a preservative. The pharmaceutical formulation of the present invention optionally comprises a preservative. Many well known preservatives that have been used in some other nasal and ophthalmic preparations cannot be used in the present invention, since those preservatives are not considered safe for repeatedly ocular use, or they interact with the viscosity-enhancing agent employed herein to form a complex that reduces the bactericidal activity of the preservative. Suitable preservatives for the present invention include benzalkonium chloride, benzyl alcohol, methyl parabens, propyl parabens, and benzethonium chlorides. In one embodiment, benzalkonium chloride is included as a safe preservative; preferably, benzalkonium chloride is used with EDTA. Typically, preservatives are employed at a level of 0.001-1%, preferably, 0.005-0.25%, and most preferably 0.05-0.2% (w/v).

The pharmaceutical formulation of the present invention optionally comprises one or more taste-masking agents to mask the bitter taste of epinastine. Taste-masking agents can be sweeteners, flavoring agents, or other agents that can mask the taste of a formulation. Taste-masking agents suitable for the present invention include saccharin sodium, erythritol, aspartame, sucrose, glycerin, sorbitol, glycyrrhinic acid, or glycyrrihinate ammonium salts. A good taste-masking agent is glycyrrihinate ammonium salts. Commercially available SWEETAM™ flavor (Flavors of North America), which contains monoammonium glycyrrihinate salts pre-blended with suitable adjuvants such as sucrose and dextrose, is also suitable to be included in the present formulation. A taste-masking agent at a level of 0.001-0.5% (w/v) can be included in the fonnulation. However, the inventor has discovered that the aqueous epinastine formulation of the present invention does not cause bitter aftertaste even without including a taste-masking agent such as sweeteners or flavoring agents.

The viscosity-enhancing agent, the tonicity agent, the buffer, the taste-masking agent, and any other ingredient introduced in the formulation must have a good solubility in water, have compatibility with other components, and have mild effects on the final viscosity of the formulation. This viscosity of the formulation is important such that the formulation can be delivered as a topical nasal spray using a metered-dose nasal spray device and is filter-sterilizable. The formulation is preferably a clear solution without any precipitate.

In one embodiment, the pharmaceutical formulation comprises epinastine or its salts in an amount of 0.05-0.2% (w/v), a non-ionic tonicity agent such as propylene glycol at 1.0-1.8% (w/v), a buffer (such as sodium phosphates) at 10-25 mM, a viscosity-enhancing agent in a range of 0.05-0.5% (w/v), an optional chelating agent in a range of 0.02-0.5% (w/v), and an optional preservative in a range of 0.005-0.2% (w/v). Such an aqueous composition has a tonicity of 250-350 mOsm/kG and is formulated at pH 5-8.

In another embodiment, an aqueous pharmaceutical formulation comprises epinastine or its salts in an amount of 0.001-3% (w/v), 1-100 mM buffer suitable to maintain the pH between 5-8, 0.001-0.5% (w/v) viscosity-enhancing agent to maintain a viscosity of 1.5-10 cps, a tonicity agent to maintain a tonicity between 200-400 mOsm/kG, and a taste-masking agent at a level of 0.001-5% w/v. The formulation optionally comprises a preservative (such as benzalkonium chloride) at a level of 0.005-0.2% w/v.

The pharmaceutical formulations of the present invention are preferably stable at room temperature for at least 12 months, preferably 24 months, and more preferably 36 months. Stable, as used herein, means that epinastine maintains at least 80%, preferably 85%, 90%, or 95% of its initial activity.

The pharmaceutical formulations of the present invention can be prepared by aseptic technique or are terminally sterilized. The purity levels of all materials used in the preparation exceed 90%. The solutions of the invention are prepared by thoroughly mixing the epinastine or salts thereof, buffer(s), tonicity agent(s), viscosity-enhancing agent(s), optionally, taste-masking agent(s), chelating agent(s), complexing agent(s), solubilizing agent(s), preservative(s) and antioxidant agent(s). Examples of complexing agents are cyclodextrins, gamma-cyclodextrin, and crosspovidone. Examples of solubilizing agents are polysorbates, crernophor, and glycerin. Examples of antioxidants are tocopherol, butylated hydroxytoulene, butylated hydroxyanisole. Complexing agents, solubilizing agents, antioxidants can be added to the formulation; however, they are not essential for the formulation of the present invention.

The pharmaceutical formulation can be sterilized by filtering the formulation through a sterilizing grade filter, preferably of a 0.1 micron nominal pore size. The pharmaceutical formulation can also be sterilized by terminally sterilization using one or more sterilization techniques including but not limited to a thermal process, or a radiation sterilization process, or using pulsed light to produce a sterile formulation.

In one embodiment, the pharmaceutical formulation of the present invention is administered locally to the eye (e.g., topically, intracamerally, or via an implant) in the form of ophthalmic preparations. The pharmaceutical formulation can be combined with additional ophthalmologically acceptable viscosity enhancers, or penetration enhancers to form an ophthalmic suspension or solution. The pharmaceutical formulation is ready for use, without further dilution or any other manipulation. The pharmaceutical formulation can be administered to the eyes of a patient topically by any suitable means, but is preferably administered in the form of drops, spray or gel. For topical ophthalmic administration, one to two drops of the formulation are delivered to the surface of the eye one to three times per day according to the routine discretion of a skilled clinician.

In another embodiment, the pharmaceutical formulation of the present invention is administered locally to the nose in the form of nasal preparations. The pharmaceutical formulation can be administered to the nasal cavity of a patient topically by any suitable means, but is preferably administered in the form of drops, or spray. For topical nasal administration, one to two sprays of the formulation is delivered to the surface of the nose one to three times per day according to the routine discretion of a skilled clinician.

The pharmaceutical formulation is preferably packaged in opaque plastic containers equipped with a nasal spray pump for topical nasal delivery. While a variety of metered-dose nasal spray pumps are available for delivery of an aqueous formulation, a metered-dose nasal spray pump that can deliver a small volume spray is preferred in order to minimize the bitter taste of epinastine. This bitter taste of epinastine is due to post-nasal drip of a solution drainage following nasal administration. The use of a viscous formulation of the present invention coupled with a small volume delivery minimizes the bitter taste perception due to the epinastine. When the total volume is delivered in multiple sprays (i.e. one 140 μL spray verses two 70 μL sprays), the bitter taste is improved. The preferred range of dose volume of a nasal spray pump for delivering the present formulation is 50-100 milligrams (about 50-100 μL aqueous solution) per actuation, more preferably 50-90 milligrams (about 50-90 μL aqueous solution) per actuation, and most preferably 60-80 milligrams (about 60-80 μL aqueous solution) per actuation.

The pharmaceutical formulations of the present invention can be used to prevent or treat diseases or disorders related to allergic and inflammatory diseases of the eye and the nose. For example, the pharmaceutical formulation is useful for treating seasonal and perennial allergic rhinitis, vasomotor rhinitis, sinusitis, asthma, COPD, or emphysema.

The present invention provides a method for treating an allergic and inflammatory disease of the eye, such as allergic conjunctivitis, giant papillary conjunctivitis, blepharitis. The method comprises the steps of (a) identifying a patient suffering from an allergic and inflammatory disease of the eye, and (b) administering to the eye of the patient an effective amount of an aqueous pharmaceutical formulation comprising epinastine or an acid addition salt thereof, and a viscosity-enhancing agent. “An effective amount,” as used herein, refers to an amount effective to reduce or alleviate the symptoms of a disease.

The present invention also provides a method for treating an allergic and inflammatory disease of the nose such as allergic rhinitis and vasomotor rhinitis. The method comprises the steps of (a) identifying a patient suffering from an allergic and inflammatory disease of the nose, and (b) administering to the nose of the patient an effective amount of an aqueous pharmaceutical formulation comprising epinastine or an acid addition salt thereof, and a viscosity-enhancing agent.

The present invention further provides a preferred method for treating allergic rhinitis in a subject. The method comprises the steps of: (a) identifying a subject suffering from allergic rhinitis; and (b) administering to the nose of the subject one to two sprays at ≦115 μL per nostril per spray once or twice daily of an aqueous epinastine formulation; wherein the aqueous epinastine formulation comprises (i) 0.05-0.2% (w/v) of epinastine or an acid addition salt thereof, (ii) 0.05-0.5% (w/v) of a viscosity-enhancing agent selected from the group consisting of hydroxypropylmethylcellulose, methylcellulose, ethylcellulose, hydroxyethylcellulose, hydroxypropylcellulose, polyvinyl alcohol, polyvinyl pyrrolidone, and the combination thereof, to maintain the viscosity between 1.5-10 centipoise, and (iii) 1-2% (w/v) of a tonicity agent to maintain a tonicity between 200-400 mOsm/kG; whereby the subject perceives a minimal or no bitter taste of said epinastine and the symptoms of allergic rhinitis are reduced. “A minimal bitter taste,” as used herein, refers to an acceptable taste perceived by the subject. In this method, a preferred viscosity-enhancing agent is hydroxypropylmethylcellulose, and its preferred concentration is 0.1-0.3% (w/v). A preferred tonicity agent is propylene glycol, and its preferred concentration is 1-2, or 1-1.8% (w/v). A preferred epinastine concentration is about 0.1-0.2% (w/v), or about 0.1-0.15% (w/v). A buffer is optionally included in the formulation to maintain the pH of 5-8.

In the preferred method, the epinastine is administered to the subject using a metered-dose nasal spray pump. The target volume of the nasal spray pump, for example, is 70 μL (±15%) or 100 μL (±15%). The aqueous epinastine formulation is preferably administered at 60-85 μL per nostril per spray, or 85-115 μL per nostril per spray. The aqueous epinastine formulation can be administered one to three times a day, preferably one to two times a day, and more preferably twice a day.

The invention is illustrated further by the following examples that are not to be construed as limiting the invention in scope to the specific procedures described in them.

### EXAMPLES

### Example 1

TABLE 1

| Ingredient | Concentration (% w/v) |
|---|---|
| Epinastine Hydrochloride | 0.6 |
| Sodium phosphate monobasic | 0.084 |
| Sodium phosphate dibasic | 0.256 |
| Disodium edetate | 0.05 |
| Benzalkonium chloride | 0.01 |
| Glycerin | 2.1 |
| Purified Water qs | 100% |

This solution was prepared by admixing the agents and filtering the resultant solution though an appropriate filter. The solution so prepared was clear, colorless, and isotonic, within the physiological pH range and had a viscosity of approximately 1 cps.

### Example 2

TABLE 2

| Ingredient | Concentration (% w/v) |
|---|---|
| Epinastine Hydrochloride | 0.6 |
| Sodium phosphate monobasic | 0.084 |
| Sodium phosphate dibasic | 0.256 |
| Disodium edetate | 0.05 |
| Benzalkonium chloride | 0.01 |
| Propylene glycol | 1.5 |
| Mixture of microcrystalline cellulose and | 0.75 |
| carboxymethyl cellulose (FMC Biopolymer) | |
| Purified Water qs | 100% |

This formulation was prepared by admixing the agents, with the exception of epinastine hydrochloride, to produce a colorless homogenous dispersion. Then epinastine hydrochloride is added to this dispersion and vigorously mixed in a vortex mixer or a homogenizer. The preparation was a viscous, cloudy, off-white preparation with floccules, which indicates incompatibility of epinastine with the polymer microcrystalline cellulose and carboxymethyl cellulose at 0.75% w/v. The preparation was isotonic, and within the physiological pH range. In light of the flocculation of the preparation, a reliable viscosity could not be obtained, it is estimated that the formulation had a viscosity of approximately 10 cps.

In a separate experiment, the concentration of a mixture of microcrystalline cellulose and carboxymethyl cellulose was lowered to 0.2% w/v and the preparation still showed floccules. The result indicates the incompatibility of epinastine with the mixture of microcrystalline cellulose and carboxymethyl cellulose even at 0.2% w/v.

### Example 3

TABLE 3

| Ingredient | Concentration (% w/v) |
|---|---|
| Epinastine Hydrochloride | 0.6 |
| Sodium phosphate monobasic | 0.084 |
| Sodium phosphate dibasic | 0.256 |
| Disodium edetate | 0.05 |
| Benzalkonium chloride | 0.01 |
| Propylene glycol | 2 |
| Hydroxypropyl methyl cellulose | 0.1 |
| Purified Water qs | 100% |

This formulation was prepared by admixing the agents, with the exception of epinastine hydrochloride, to produce a colorless homogenous viscous solution. Then epinastine hydrochloride was added to this dispersion and vigorously mixed in a vortex mixer or a homogenizer. The preparation was a viscous, clear, colorless preparation, and was easy to be filtered using a standard filtration apparatus. The preparation was isotonic, and within the physiological pH range. The formulation had a viscosity of approximately 3 cps.

### Example 4

TABLE 4

| Ingredient | Concentration (% w/v) |
|---|---|
| Epinastine Hydrochloride | 0.3 |
| Sodium phosphate monobasic | 0.084 |
| Sodium phosphate dibasic | 0.256 |
| Disodium edetate | 0.05 |
| Benzalkonium chloride | 0.01 |
| Propylene glycol | 2 |
| Hydroxypropyl methyl cellulose | 0.3 |
| Purified Water qs | 100% |

This formulation was prepared by admixing the agents, with the exception of epinastine hydrochloride, to produce a colorless homogenous viscous solution. Then epinastine hydrochloride was added to this dispersion and vigorously mixed in a vortex mixer or a homogenizer. The preparation was a viscous, clear, colorless preparation, and was easy to be filtered using a standard filtration apparatus. The preparation was isotonic, and within the physiological pH range. The formulation had a viscosity of approximately 8 cps.

### Example 5

TABLE 5

| Ingredient | Concentration (% w/v) |
|---|---|
| Epinastine Hydrochloride | 0.3 |
| Sodium phosphate monobasic | 0.084 |
| Sodium phosphate dibasic | 0.256 |
| Disodium edetate | 0.05 |
| Benzalkonium chloride | 0.01 |
| Propylene glycol | 2 |
| Hydroxypropyl methyl cellulose | 0.1 |
| SWEETAM ™ | 0.005 |
| Purified Water qs | 100% |

This formulation is prepared by admixing the agents, with the exception of epinastine hydrochloride, to produce a colorless homogenous viscous solution. Then epinastine hydrochloride is added to this dispersion and vigorously mixed in a vortex mixer or a homogenizer. The preparation was a viscous, clear, colorless preparation, and was easy to be filtered using a standard filtration apparatus. The preparation was isotonic, and within the physiological pH range. The formulation had a viscosity of approximately 3 cps.

### Example 6

### Clinal Trial Results

A Phase 2 clinical trial involving a 14-day, randomized, double-blind comparison of two doses of epinastine nasal spray (0.05% or 0.1%) or placebo in subjects who had a documented history of seasonal allergic rhinitis to mountain cedar pollen was conducted. Each group started with approximately 190 subjects. Each subject was administered twice daily, two sprays per nostile (total 4 sprays per administration), of a target volume of 70 μL/spray of placebo, 0.05%, or 0.1% epinastine formulation (see Table 6), using a metered-dose spray pump for 14 days. The epinastine formulations did not contain any taste-masking agent.

TABLE 6
Epinastine nasal spray formulation for the clinical studies

| | Epinastine | Epinastine | Epinastine |
|---|---|---|---|
| | nasal | nasal spray, | nasal spray, |
| | spray, | 0.5 mg/mL | 1 mg/mL |
| Formulation Name | Placebo | (0.05%) | (0.1%) |
| Component (% w/v) | | | |
| Epinastine hydrochloride | 0.0 | 0.05 | 0.1 |
| Hypromellose | 0.1 | 0.1 | 0.1 |
| Propylene Glycol | 1.6 | 1.5 | 1.5 |
| Sodium phosphate, | 0.084 | 0.084 | 0.084 |
| monobasic, anhydrous | | | |
| Sodium phosphate, dibasic, | 0.256 | 0.256 | 0.256 |
| anhydrous | | | |
| Disodium edetate | 0.05 | 0.05 | 0.05 |
| Benzalkonium chloride | 0.01 | 0.01 | 0.01 |
| Purified Water | QS | QS | QS |
| Adjust pH to | 7.0 ± 0.5 | 7.0 ± 0.5 | 7.0 ± 0.5 |

### Taste

Of the subjects randomized in the trial, 95% completed the trial and no serious adverse events were reported. While the most common adverse event observed was bitter taste, it was only reported by 4% of subjects in the 0.05% group and by 5% of subjects in the 0.1% group. The results demonstrated a low incidence of taste complaints with the two doses evaluated and showed acceptable tolerability of the formulation.

### Efficacy of Treatment

The primary endpoint of the trial was the daily reflective change from baseline for total nasal symptom score (TNSS), averaged over the 14-day treatment period. The endpoint of TNSS conforms to the U.S. Food and Drug Administration's (FDA) draft guidance document for seasonal allergic rhinitis and includes runny nose, nasal congestion, itchy nose and sneezing. The TNSS is the sum of four nasal symptom scores (runny nose, nasal congestion, itchy nose, and sneezing), each evaluated by the subject on a 0-3 scale. The TNSS can range from 0 to 12 total points. Results of the trial demonstrated statistically significant improvement (p<0.05) in reflective TNSS for the 0.1% dose group, compared to placebo. Changes in TNSS for the 0.05% dose group were not statistically significant.

The non-nasal symptoms were combined to generate the non-nasal symptom score (NNSS). The NNSS is the sum of three non-nasal symptom scores (itchy throat/palate, itchy eyes and watery eyes), each evaluated by the subject on a 0-3 scale. The NNSS can range from 0 to 9 total points. Total Symptom Score (TSS) is the sum of TNSS and NNSS and can range from 0-21 total points.

There were multiple secondary endpoints in this trial. The secondary endpoints include change from baseline for both NNSS and TSS, change from baseline for individual symptom scores, change from baseline in the night-time symptom score, and change from baseline in quality of life as assessed by the self-administered standardized Rhino conjunctivitis Quality of Life Questionnaire. Among these, statistically significant improvements (p<0.05) compared to placebo were demonstrated in the 0.1% epinastine dose group for the secondary endpoints of non-nasal symptom score (NNSS), TSS, and individual symptoms of runny nose, itchy nose, sneezing, itchy eyes, and watery eyes.

The plasma samples from 43 subjects included in this clinical trial were collected for pharamacokinetic analysis following the first dose (Day 1) and the last dose (Day 14). Plasma epinastine levels following intranasal administration were determined. The plasma epinastine concentrations were generally proportional to the nasal dose administered. The plasma concentrations of epinastine were <1 ng/mL, which was well below those required to produce systemic pharmacological effects by an oral administration. The nasal delivery of the current formulation using a small volume metered-dose nasal spray pump produced low, dose-proportional systemic levels of epinastine.

### Example 7

### Comparison of Taste Preference of Epinastine Formulations with and without a Taste-Masking Agent

### Objectives

There were no efficacy parameters being measured in this study. This study was a taste preference study only. The assessments of taste preference were the individual sensory attribute scores (0 to 100 scale) from the Nasal Spray Evaluation Questionnaire (NSEQ) and a stated overall preference rank for each treatment from the Overall Nasal Spray Evaluation Questionnaire (ONSEQ).

### Evaluation Criteria

The NSEQ consisted of 14 individual questions scored on a scale from 0 (least satisfaction) to 100 (greatest satisfaction) and was reported by subjects at 0-5 minutes, 30 minutes, and 90 minutes after each study drug administration. The individual questions asked were as follows. Question 1: How uncomfortable is it to use this nasal spray? Question 2: How much medicine ran down your throat? Question 3: How much medicine ran out of your nostrils? Question 4: Rate the urge to sneeze. Question 5: Rate the smell of the nasal spray. Question 6: Rate your satisfaction of the nasal spray smell. Question 7: Rate the taste of the nasal spray. Question 8: Rate your satisfaction of the nasal spray taste. Question 9: Rate the bitterness of the nasal spray. Question 10: Rate the aftertaste of the nasal spray. Question 11: Rate your satisfaction of the nasal spray aftertaste. Question 12: Rate the dryness of your nose. Question 13: Rate the dryness of your throat. (0=Extremely dry; 100=Not at all dry) Question 14: Rate your overall satisfaction of this product based on your assessment of questions 1-13. The ONSEQ asked for patient's overall taste preference and was assessed by subjects at 90 minutes after the last (3rd) study drug administration.

### Susbjects

Subjects were 18-70 years of age with a history of seasonal allergic rhinitis (SAR) or perennial allergic rhinitis (PAR) and must have had an instantaneous symptom score total of <4 on the Total Nasal Symptom Score (TNSS) questionnaire (see Example 6). This served to recruit subjects with allergic rhinitis who had mild to no symptoms.

### Test Protocols

This was a double-blind, randomized, cross-over, taste preference study. Subjects were randomized into one of two cohorts. Cohorts 1 and 2 received epinastine in concentrations of 0.1% and 0.2%, respectively. In each cohort, subjects received a single dose of epinastine without SWEETAM™, epinastine with SWEETAM™, and a non-epinastine formulation in a random order. Each dose administration was delivered as two sprays (70 μL/spray) in each nostril (4 sprays total per dosing) and each dosing period was separated by at least 90 minutes (up to 120 minutes). The 0.1% epinastine formulation (Formulation A) without SWEETAM™ was identical to that as described in Table 6. The 0.1% epinastine formulation with SWEETAM™ (Formulation B) was identical to Formulation A except it additionally contains SWEETAM™ at 0.05% w/v. The 0.2% epinastine formulation (Formulation C) without SWEETAM™ was identical to Formulation A except it contained 0.2% epinastine. The 0.2% epinastine formulation (Formulation D) with SWEETAM™ was identical to Formulation B except it contained 0.2% epinastine.

After each dose administration, subjects completed the NSEQ, examining the sensory attributes of each nasal spray 5, 30, and 90 minutes post dose. At the end of the study (following the third dose of study drug), the Overall Nasal Spray Evaluation Questionnaire (ONSEQ) was given to rank the taste preference. The ONSEQ was administered following NSEQ completion at the 85-95 minute post dose time-point after Dose 3.

### Conclusions

Epinastine unmasked (without SWEETAM™) and epinastine masked (with SWEETAM™) formulations had similar sensory attribute ratings and were well accepted with regard to the taste preference. There was no consistent evidence that suggested that there were any benefits with respect to overall preference or nasal spray attributes associated with the use of masked formulation of epinastine compared with the unmasked formulation of epinastine.

The invention, and the manner and process of making and using it, are now described in such full, clear, concise and exact terms as to enable any person skilled in the art to which it pertains, to make and use the same. It is to be understood that the foregoing describes preferred embodiments of the present invention and that modifications may be made therein without departing from the scope of the present invention as set forth in the claims. To particularly point out and distinctly claim the subject matter regarded as invention, the following claims conclude this specification.

1. A method for treating allergic rhinitis in a subject, comprising the steps of:
identifying a subject suffering from allergic rhinitis;
administering to the nose of the subject one to two sprays at≦115 μl per nostril per spray once or twice daily of an aqueous epinastine formulation;
wherein said aqueous epinastine formulation comprises (a) 0.1-0.15% (w/v) of epinastine or an acid addition salt thereof, (b) 0.05-0.5% (w/v) of hydroxypropylmethylcellulose to maintain the viscosity between 1.5-10 centipoise, (c) 1-2% (w/v) of propylene glycol, and (d) a buffer to maintain the pH between 5-8, said aqueous epinastine formulation has a tonicity between 200-400 mOsm/kG and does not include a sweetening agent;
whereby the subject perceives a minimal or no bitter taste of said epinastine and the symptoms of allergic rhinitis are reduced.

2-3. (canceled)

4. The method according to claim 1, wherein said aqueous epinastine formulation further comprises a buffer at 1-100 mM to maintain a pH between 5-8.

5. The method according to claim 1, wherein the aqueous epinastine formulation is administered at 60-85 μl per nostril per spray.

6. The method according to claim 5, wherein the aqueous epinastine formulation is administered at about 70 μl per nostril per spray.

7. The method according to claim 1, wherein the aqueous epinastine formulation is administered at 85-115 μl per nostril per spray.

8. The method according to claim 7, wherein the aqueous epinastine formulation is administered at about 100 μl per nostril per spray.

9. (canceled)

10. The method according to claim 1, wherein said aqueous epinastine formulation further comprises a chelating agent.

11. The method according to claim 1, wherein said aqueous epinastine formulation further comprises a preservative.

12. (canceled)

13. The method according to claim 1, wherein said aqueous epinastine formulation is administered twice daily.

14-15. (canceled)

16. The method according to claim 1, wherein said aqueous epinastine formulation comprises 0.1-0.3% (w/v) of hydroxypropylmethylcellulose.

17-19. (canceled)

20. The method according to claim 1, wherein the allergic rhinitis is seasonal allergic rhinitis, perennial allergic rhinitis, or vasomotor rhinitis.
